(19) United States
(12) Patent Application Publication
(10) Pub. No.: US 2019/0204314 A1
(43) Pub. Date: Jul. 4, 2019

(54) SAMPLE ANALYSIS CARTRIDGE AND SAMPLE ANALYSIS APPARATUS HAVING THE SAME

(71) Applicant: SAMSUNG ELECTRONICS CO., LTD., Suwon-si (KR)

(72) Inventors: Seung Hyun KIM, Anyang-si (KR); Kyu Youn HWANG, Sejong-si (KR)

(73) Assignee: SAMSUNG ELECTRONICS CO., LTD., Suwon-si (KR)

(21) Appl. No.: 16/057,206

(22) Filed: Aug. 7, 2018

(30) Foreign Application Priority Data
Jan. 2, 2018 (KR) 10-2018-0000359

(51) Int. Cl. G01N 33/543

(52) U.S. Cl. CPC G01N 33/54386

(57) ABSTRACT

A sample analysis cartridge and a sample analysis apparatus having the sample analysis cartridge are provided. The sample analysis cartridge includes a housing having a sample injecting hole and a strip coupled with the housing such that a sample that is passed through the sample injecting hole is directed into the strip, and the strip is configured to detect a target material from the sample through an antigen-antibody reaction. The strip includes a membrane including a test line and a transparent cover disposed outside the membrane.

### CROSS-REFERENCE TO RELATED APPLICATION

This application is based on and claims priority under 35 U.S.C. § 119 to Korean Patent Application No. 10-2018-0000359, filed on Jan. 2, 2018, in the Korean Intellectual Property Office, the disclosure of which is incorporated by reference herein in its entirety.

### BACKGROUND

### 1. Field

The disclosure relates to a sample analysis cartridge, and a sample analysis apparatus having the same, and more particularly, to a sample analysis cartridge having an improved structure for securing reliability and speed of a test, and a sample analysis apparatus having the sample analysis cartridge.

### 2. Description of the Related Art

An apparatus and a method for analyzing fluidic samples are needed in various fields, such as environmental monitoring, food inspection, and medical diagnosis. Typically, in order to perform a test according to a given protocol, a skilled experimenter manually performs various steps including injection of reagents, mixing, separation and transfer, reaction, and centrifugation. However, sometimes, errors can be caused when conducting the test if the steps are manually performed.

In order to overcome this problem, miniaturized and automated equipment has been developed to analyze a test material quickly. Particularly, a portable sample analysis cartridge is expected to perform more functions in more various fields through an improvement in structure and function, since it can analyze fluidic samples quickly anywhere. Accordingly, there is a need for research and development to produce a portable sample analysis cartridge with such characteristics. Also, such a portable sample analysis cartridge has an advantage that unskilled persons can easily perform a test.

### SUMMARY

Therefore, it is an aspect of the disclosure to provide a sample analysis cartridge having an improved structure for detecting a target material from a sample using an immune response, and a sample analysis apparatus having the sample analysis cartridge.

It is another aspect of the disclosure to provide a sample analysis cartridge having an improved structure capable of performing quantitative analysis, as well as qualitative analysis, on a sample, and a sample analysis apparatus having the sample analysis cartridge.

Additional aspects will be set forth in part in the description which follows and, in part, will be apparent from the description, or may be learned by practice of the presented embodiments.

In accordance with an aspect of the disclosure, a sample analysis cartridge may include a housing including a sample injecting hole and a strip coupled with the housing and arranged such that a sample that is passed through the sample injecting hole is directed into the strip, and the strip is configured to detect a target material from the sample through an antigen-antibody reaction. The strip may include a membrane including a test line and a transparent cover disposed outside the membrane.

The housing may further include a first frame in which the sample injecting hole is formed and a second frame coupled with the first frame to form an outer appearance of the housing. A portion of the strip may be coupled with the housing such that an area or portion of the strip is located between the first frame and the second frame.

The second frame includes a recessed portion on which the strip may be disposed.

The strip may further include an absorptive pad disposed around the membrane and is configured to absorb the sample that is passed through the membrane.

The membrane may include a first end located adjacent to the sample injecting hole and a second end that is opposite to the first end. The absorptive pad may be disposed around the second end of the membrane.

The strip may include a sample pad facing the sample injecting hole, and configured to absorb the sample that is passed through the sample injecting hole and a binding pad disposed between the sample pad and the membrane, and having an indicator configured to be bound with the target material of the sample.

The indicator may include at least one from among a gold nanoparticle and a latex particle.

The housing may further include a sample supplier protruding a surface of the housing to have a predetermined height, and the sample injecting hole is formed in the sample supplier.

The strip may include a plurality of membranes. The housing may include a sample accommodating portion formed in an inner surface of the housing to accommodate the sample that is passed through the sample injecting hole and a sample moving channel connecting the sample accommodating portion to the plurality of membranes to transfer the sample accommodated in the sample accommodating portion to the respective membranes.

The sample accommodating portion may be partitioned into a plurality of sample accommodating spaces by a partition wall.

The partition wall may protrude from an inner surface of the housing.

In accordance with another aspect of the disclosure, a sample analysis cartridge may include a transparent housing including a sample injecting hole, and made of a transparent material and a strip disposed in the inside of the housing and arranged such that a sample that is passed through the sample injecting hole is directed to the strip, and the strip is configured to detect a target material from the sample through an antigen-antibody reaction.

The transparent housing may further include a first frame in which the sample injecting hole is formed, a second frame coupled with the first frame to form an outer part of the housing, and comprising a recessed portion on which the strip is disposed.

The strip may include a membrane including a test line and a transparent base cover configured to support the membrane, and made of a transparent material.

The strip may further include an absorptive pad overlapping the membrane, and configured to absorb the sample that is passed through the membrane.

The strip may further include a sample pad facing the sample injecting hole, and configured to absorb the sample that is passed through the sample injecting hole and a binding pad disposed between the sample pad and the membrane, and having an indicator that can bind with the target material of the sample.

The indicator may be at least one from among a gold nanoparticle and a latex particle.

In accordance with still another aspect of the disclosure, a sample analysis apparatus may include a sample analysis cartridge including a housing including at least one sample injecting hole, and a strip coupled with the housing such that a sample that is passed through the sample injecting hole is directed into the strip, the strip having a test line configured to detect a target material from the sample through an antigen-antibody reaction, a pressing member configured to press and seal off the at least one sample injecting hole and an optical measurement module configured to measure optical characteristics of the test line.

The optical measurement module may include a light emitter configured to radiate light to the test line and a light receiver configured to receive the light that passes through the test line. At least one area of the strip corresponding to the test line may be made of a light transmitting material.

The strip may include at least one membrane underneath the at least one sample injecting hole, and including the test line, an absorptive pad disposed around the at least one membrane, and configured to absorb the sample that is passed through the at least one membrane and a transparent cover disposed outside the at least one membrane and the absorptive pad, and configured to support the at least one membrane and the absorptive pad, and made of a transparent material.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a perspective view showing an outer appearance of a sample analysis apparatus according to an embodiment;

FIG. 2 is a perspective view of a sample analysis cartridge according to a first embodiment;

FIG. 3 is an exploded perspective view of the sample analysis cartridge according to the first embodiment;

FIG. 4 is an exploded perspective view of a strip included in the sample analysis cartridge according to the first embodiment;

FIG. 5 is a cross-sectional view of the sample analysis cartridge according to the first embodiment;

FIGS. 6A and 6B show various arrangements of an absorptive pad, in a sample analysis cartridge according to a first embodiment;

FIG. 7 is a perspective view of a sample analysis cartridge according to a second embodiment;

FIG. 8 is an exploded perspective view of the sample analysis cartridge according to the second embodiment;

FIGS. 9A, 9B, and 9C show various arrangements of an absorptive pad, in the sample analysis cartridge according to the second embodiment;

FIG. 10 is a perspective view of a sample analysis cartridge according to a third embodiment;

FIG. 11 is an exploded perspective view of the sample analysis cartridge according to the third embodiment;

FIG. 12 is a perspective view of a sample analysis cartridge according to a fourth embodiment;

FIG. 13 is a cross-sectional view of the sample analysis cartridge according to the fourth embodiment, and

FIG. 14 is a view describing an example process for measuring optical characteristics of the sample analysis cartridge according to the first embodiment.

### DETAILED DESCRIPTION

Hereinafter, embodiments will be described in detail with reference to the appended drawings. In the following description, the terms “front end”, “rear end”, “upper portion”, “lower portion”, “upper end”, and “lower end” are defined based on the drawings, and the shapes and positions of the corresponding components are not limited by the terms. Expressions such as “at least one of” and “at least one from among,” when preceding a list of elements, modify the entire list of elements and do not modify the individual elements of the list. For example, the expression, “at least one of a, b, and c” or “at least one from among a, b, and c,” should be understood as including only a, only b, only c, both a and b, both a and c, both b and c, or all of a, b, and c.

A sample that can be analyzed by a sample analysis apparatus and a sample analysis cartridge according to the disclosure may be a fluidic sample. The fluidic sample may be a biological sample including bodily fluids (for example, blood, tissue fluid, and/or lymphatic fluid), saliva, and urine, or an environmental sample for water-quality management or soil management. The fluidic sample may be a compound or a composite having one or more epitopes, binding sites, or ligands, which is detected or measured. The fluidic sample may be toxins, an organic compound, proteins, peptide, microbes, amino acids, nucleic acids, hormones, steroid, vitamin, drugs, or a metabolite or antibody of any one of the above-mentioned materials, although not limited thereto. Also, the fluidic sample may be a specific antigenic material, a hapten, an antibody, a giant molecule, or a combination thereof.

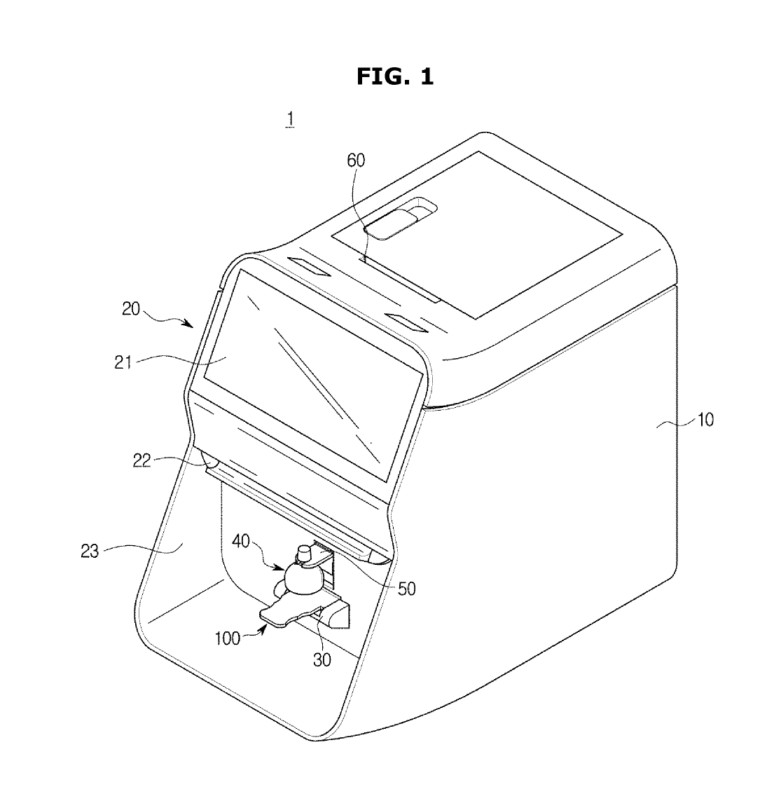

FIG. 1 is a perspective view showing an outer appearance of a sample analysis apparatus according to an embodiment.

As shown in FIG. 1, a sample analysis apparatus 1 may include a casing 10 forming an outer appearance, and a door module 20 disposed in a front portion of the casing 10.

The door module 20 may include a display 21, a door 22, and a door frame 23. The display 21 and the door 22 may be disposed in a front portion of the door frame 23. The display 21 may be located above the door 22. The door 22 may be configured to slide, and when the door 22 slides to open, the door 22 may be positioned behind the display 21.

The display 21 may be a liquid crystal display (LCD) or the like that displays information about the results of sample analysis, an operation state for sample analysis, etc. The door frame 23 may include an installation member 30 on which a sample analysis cartridge 100 can be installed. A user may push up the door 22 to open the door 22, then install the sample analysis cartridge 100 on the installation member 30, push down the door 22 to close the door 22, and thereafter, perform an analysis operation.

The sample analysis apparatus 1 may further include the sample analysis cartridge 100.

The sample analysis cartridge 100 may be removably coupled with the sample analysis apparatus 1.

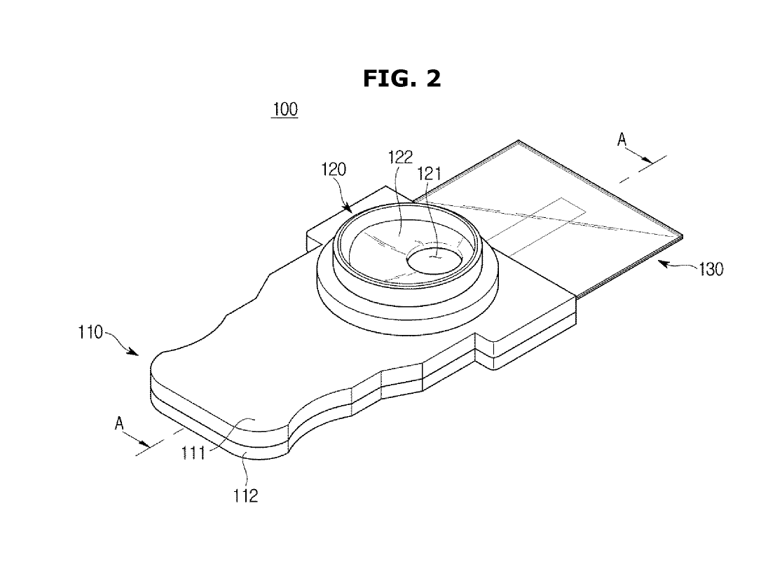

The sample analysis apparatus 1 may further include a pressing member 40 for pressing the sample analysis cartridge 100. The pressing member 40 may be coupled with a lever 50 of the sample analysis apparatus 1. The pressing member 40 may press and seal off a sample injecting hole 121 (see FIG. 2) of the sample analysis cartridge 100. The pressing member 40 may be made of an elastic material.

The sample analysis cartridge 100 may be inserted into the installation member 30, and the pressing member 40 may press the sample analysis cartridge 100 to cause a sample contained in the sample analysis cartridge 100 to enter a strip 130 (see FIG. 2).

The sample analysis apparatus 1 may further include an output device 60 for outputting the results of a test as printouts, separately from the display 21. The output device may be a printer for printing test results on a recording medium such as paper.

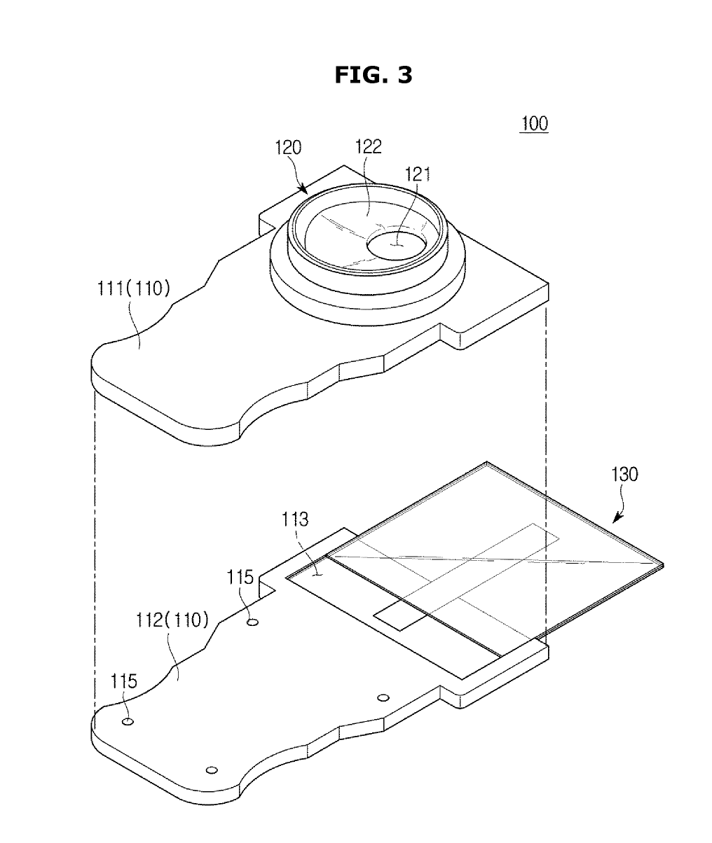

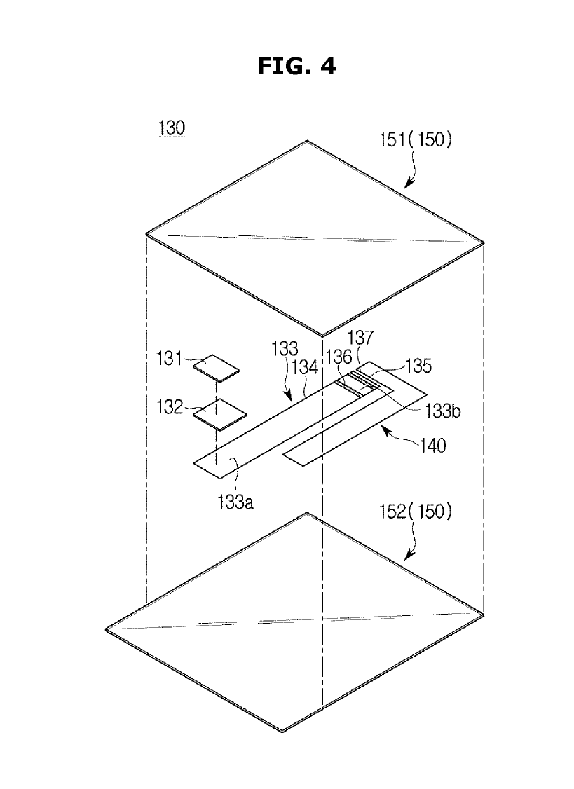

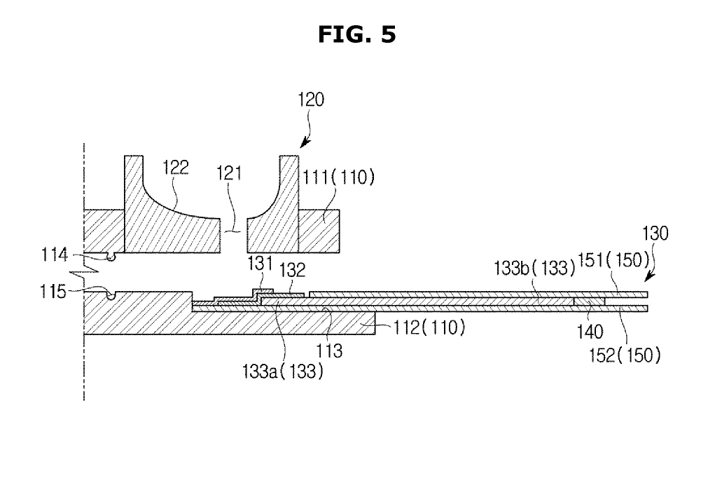

FIG. 2 is a perspective view of a sample analysis cartridge according to a first embodiment, FIG. 3 is an exploded perspective view of the sample analysis cartridge according to the first embodiment, FIG. 4 is an exploded perspective view of a strip included in the sample analysis cartridge according to the first embodiment, and FIG. 5 is a cross-sectional view of the sample analysis cartridge according to the first embodiment. More specifically, FIG. 5 is a cross-sectional view of the sample analysis cartridge 100 of FIG. 2, taken along line A-A of FIG. 2.

As shown in FIGS. 2 to 5, the sample analysis cartridge 100 may include a housing 110.

The housing 110 may include a first frame 111, and a second frame 112 coupled with the first frame 111 to form an outer appearance of the housing 110.

On any one of the first frame 111 and the second frame 112, a coupling protrusion 114 may be formed. In the other one of the first frame 111 and the second frame 112, a coupling groove 115 corresponding to the coupling protrusion 114 may be formed. The coupling protrusion 114 may be coupled with the coupling groove 115 so that the first frame 111 can be coupled with the second frame 112.

However, the first frame 111 may be coupled with the second frame 112 by another method. For example, the first frame 111 may be coupled with the second frame 112 by an adhesive material or member. Also, the first frame 111 may be coupled with the second frame 112 by welding.

The housing 110 may further include a grip portion to enable the user to easily grip the sample analysis cartridge 100. The grip portion may be formed in the shape of a streamlined protrusion to enable the user to stably grip the sample analysis cartridge 100.

The housing 110 may further include a resting portion 113 on which the strip 130 can be rested. The resting portion 113 may be recessed in the inner surface of the housing 110. More specifically, the resting portion 113 may be a recessed portion that is recessed in the inner surface of the second frame 112.

The sample analysis cartridge 100 may further include a sample supplier 120 for supplying a sample. The sample supplier 120 may be formed in the housing 110. Specifically, the sample supplier 120 may protrude from the housing 110 to have a predetermined height. More specifically, the sample supplier 120 may protrude from the first frame 111 of the housing 110 to have a predetermined height. The sample supplier 120 may protrude from the first frame 111 of the housing 110 such that when the sample analysis cartridge 100 is inserted in the installation member 30, the sample supplier 120 faces the pressing member 40. The sample supplier 120 may include a sample injecting hole 121 for supplying a sample into the strip 130, and a sample injection guide 122 for guiding the supply of the sample.

The sample injecting hole 121 may be in the shape of a circle, although its shape is not limited thereto. According to another example, the sample injecting hole 121 may be in the shape of a polygon. The user may drop a sample on the sample supplier 120 using a tool, such as pipet or a syringe. The sample injection guide 122 may be formed around the sample injecting hole 121 in such a way as to be inclined toward the sample injecting hole 121. Accordingly, the sample dropped around the sample injecting hole 121 may flow into the sample injecting hole 121 along the inclination of the sample injection guide 122. More specifically, when the user fails to drop a sample exactly into the sample injecting hole 121 so that the sample falls around the sample injecting hole 121, the sample fallen around the sample injecting hole 121 may flow down due to the inclination of the sample injection guide 122 and the effect of gravity so as to enter the sample injecting hole 121.

The sample analysis cartridge 100 may further include the strip 130 into which a sample entered through the sample injecting hole 121 is supplied. The strip 130 may be coupled with the housing 110. More specifically, the strip 130 may be coupled with the housing 110 such that at least one area of the strip 130 is located in the inside of the housing 110. For example, the strip 130 may be coupled with the housing 110 such that at least one area of the strip 130 is positioned between the first frame 111 and the second frame 112. That is, the strip 130 may be disposed on rested on the resting portion 113 of the housing 110 such that an area of the strip 130 is located between the first frame 111 and the second frame 112.

The strip 130 may detect a target material from a sample through an antigen-antibody reaction.

The strip 130 may include a sample pad 131 for absorbing the sample entered through the sample injecting hole 121. The sample pad 131 may be disposed in the inside of the housing 110 to face the sample injecting hole 121. The sample pad 131 may be made of a porous material to sufficiently absorb the sample. For example, the sample pad 131 may be made of fibrous paper, a micropore membrane made of a cellulose material such as paper, a cellulose derivate such as cellulose and cellulose acetate, nitrocellulose, glass fiber, natural cotton, fabric such as nylon, or porous gel. However, the sample pad 131 may be made from other materials and is not limited to the above-mentioned examples.

The strip 130 may further include a binding pad 132 disposed at the sample pad 131 to transfer the sample absorbed in the sample pad 131. The binding pad 132 may be disposed between the sample pad 131 and a membrane 133. The binding pad 132 may be disposed in the inside of the housing 110, together with the sample pad 131. The binding pad 132 may contact the sample pad 131 and the membrane 133. The binding pad 132 may be made of a porous material to sufficiently absorb the sample transferred from the sample pad 131.

The binding pad 132 may include a conjugate. The conjugate may sink in the binding pad 132 to spread. The conjugate may include an indicator and a first binder that is specifically bound with the target material of the sample, wherein the first binder may be conjugated with the indicator. The target material of the sample may be bound with the conjugate in the binding pad 132 to form a first immune conjugate. The indicator may be at least one of a gold nanoparticle and a latex particle.

The term “conjugate” may refer to a combination of a specific binding component, and a detectable indicator bound with the specific binding component. The specific binding component (or, the binder) may be bound with the indicator by covalent binding or noncovalent binding. The indicator may generate a detectable signal related directly or indirectly to an amount of the target material in the sample. The specific binding component or the binder included in the conjugate may be selected to be specifically bound with the target material of the sample. The specific binding component or the binder may be an antigen, an antibody, a hapten, or a compound thereof. The antibody may be a monoclonal antibody, a polyclonal antibody, recombinant protein, or a chimeric antibody.

The strip 130 may further include the membrane 133. A sample supplied to the membrane 133 may move along the membrane 133 by a capillary phenomenon. The membrane 133 may be disposed at the binding pad 132 to transfer the sample supplied to the binding pad 132. In other words, the membrane 133 may contact the binding pad 132. The membrane 133 may include a first end 133a located adjacent to the sample injecting hole 121, and a second end 133b that is opposite to the first end 133a. The first end 133a of the membrane 133 may be located in the inside of the housing 110, and the second end 133b of the membrane 133 may be located outside the housing 110. The binding pad 132 may be applied on the first end 133a of the membrane 133.

The membrane 133 may include a test line 136. On the test line 136, a second binder that is specifically bound with the target material of the sample may be fixed. The first binder of the binding pad 132 may move together with the sample, whereas the second binder of the test line 136 may be fixed on the test line 136 without moving together with the sample. The first immune conjugate may be bound with the second binder on the test line 136 to form a second immune conjugate. The second immune conjugate may be fixed on the test line 136. Thereby, the target material of the sample may be bound between the first binder and the second binder in the test line 136. For example, the target material of the sample may be sandwiched between the first binder and the second binder in the test line 136. The second binder fixed on the test line 136 may be attached to the target material of the sample, and the indicator may also be fixed on the test line 136. Since an amount of the indicator fixed on the test line 136 is proportional to an amount of the target material included in the sample, an amount of the target material existing in the sample can be accurately measured by measuring an amount of the indicator existing on the test line 136.

The membrane 133 may further include a control line 137. The control line 137 may be located downstream from the test line 136 in a direction in which the sample entered through the sample injecting hole 121 moves. A conjugate not bound with the target material of the sample in the binding pad 132 may pass through the test line 136, together with the sample. The binding pad 132 may include a sufficiently large amount of conjugates compared to an amount of the target material estimated to be contained in the sample. The conjugate passed through the test line 136 may be fixed on the control line 137 of the membrane 133. That is, the control line 137 may include a third binder that is specifically bound with the first binder, not with the target material of the sample. The third binder may be fixed on the control line 137 such that it does not flow together with the sample. The control line 137 may inform whether the sample entered through the sample injecting hole 121 has moved until the control line 137. That is, the control line 137 may inform whether the sample entered through the sample injecting hole 121 has passed through the test line 136.

The strip 130 may further include an absorptive pad 140 disposed around the membrane 133 to absorb the sample passed through the membrane 133. That is, the absorptive pad 140 may be disposed around the membrane 133 to absorb the sample passed sequentially through the test line 136 and the control line 137. The absorptive pad 140 may be located downstream from the membrane 133 in the direction in which the sample entered through the sample injecting hole 121 moves. The absorptive pad 140 may be disposed around the second end 133b of the membrane 133. The absorptive pad 140 may contact the second end 133b of the membrane 133. The absorptive pad 140 and the membrane 133 may be co-planar. More specifically, the absorptive pad 140 and the membrane 133 may be disposed on a base cover 152 which will be described later. The absorptive pad 140 may be made of a porous material to absorb the sample from the membrane 133.

As shown in FIG. 4, the absorptive pad 140 may surround a plurality of corners of the membrane 133. If the membrane 133 has a rectangular shape including a longer side 134 extending along the direction in which the sample entered through the sample injecting hole 121 moves, and a shorter side 135 neighboring the longer side 134, the absorptive pad 140 may be positioned outside the longer side 134 and the shorter side 135 of the membrane 133. More specifically, the absorptive pad 140 may be positioned outside one shorter side 135 and one longer side 134 of the second end 133b of the membrane 133.

The strip 130 may further include a cover 150 for supporting the membrane 133. The cover 150 may be disposed outside the membrane 133 to define surfaces of the strip 130. The cover 150 may include a top cover 151 facing the first frame 111 of the housing 110, and a base cover 152 facing the second frame 112 of the housing 110. The base cover 152 may be disposed or rested on the resting portion 113 of the second frame 112. The top cover 151 may be disposed on the membrane 133 not so as to prevent the sample entered through the sample injecting hole 121 from being supplied to the sample pad 131. In other words, the top cover 151 may be disposed on the membrane 133 not so as to interfere with the sample pad 131 and the binding pad 132. The top cover 151 may have a shorter length than the base cover 152 in the direction in which the sample entered through the sample injecting hole 121 moves.

The cover 150 may be made of a transparent material. The cover 150 may be made of a transparent material configured to transmit light. For example, the cover 150 may be made of a polyethylene terephthalate (PET) film. However, the cover 150 may be made of another material, instead of a PET film.

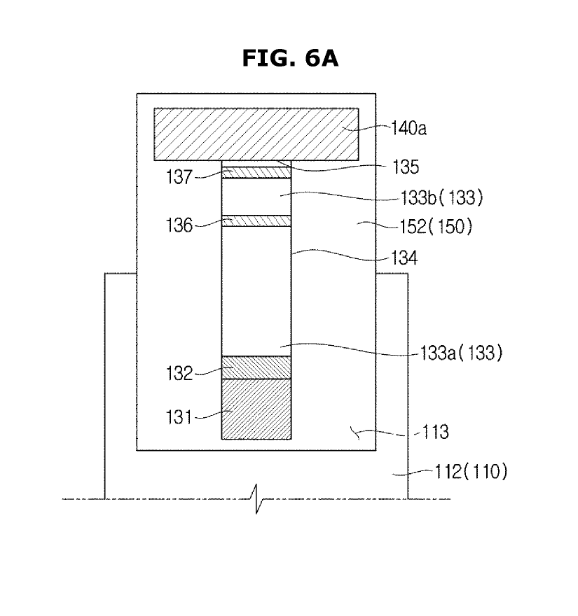

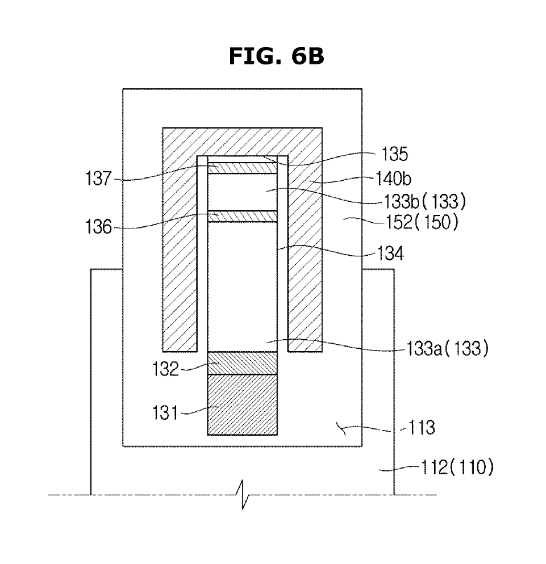

FIGS. 6A and 6B show various arrangements of an absorptive pad, in a sample analysis cartridge according to a first embodiment. Hereinafter, descriptions overlapping with those described above with reference to FIGS. 1 to 5 will be omitted.

As shown in FIG. 6A, an absorptive pad 140a may be disposed outside the membrane 133. More specifically, the absorptive pad 140a may be disposed outside the membrane 133 to face the longer side 134 or the shorter side 135 of the membrane 133. FIG. 6A shows an example in which the absorptive pad 140a is disposed outside the membrane 133 to face the shorter side 135 of the second end 133b of the membrane 133. The absorptive pad 140a may contact the longer side 134 or the shorter side 135 of the membrane 133.

As shown in FIG. 6B, the absorptive pad 140b may surround a plurality of corners of the membrane 133. More specifically, the absorptive pad 140b may be disposed outside the membrane 133 to surround the shorter side 135 of the membrane 133 and two opposite longer sides 134 of the membrane 133. In this case, the absorptive pad 140b may contact at least one of the shorter side 135 and the two longer sides 134 of the membrane 133.

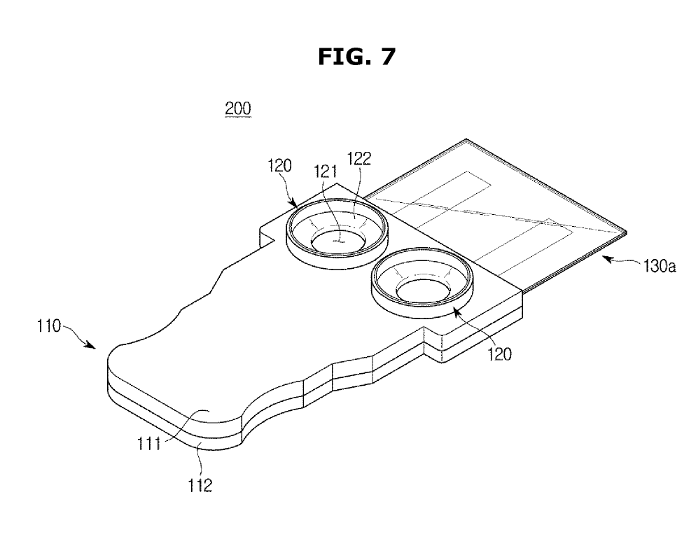

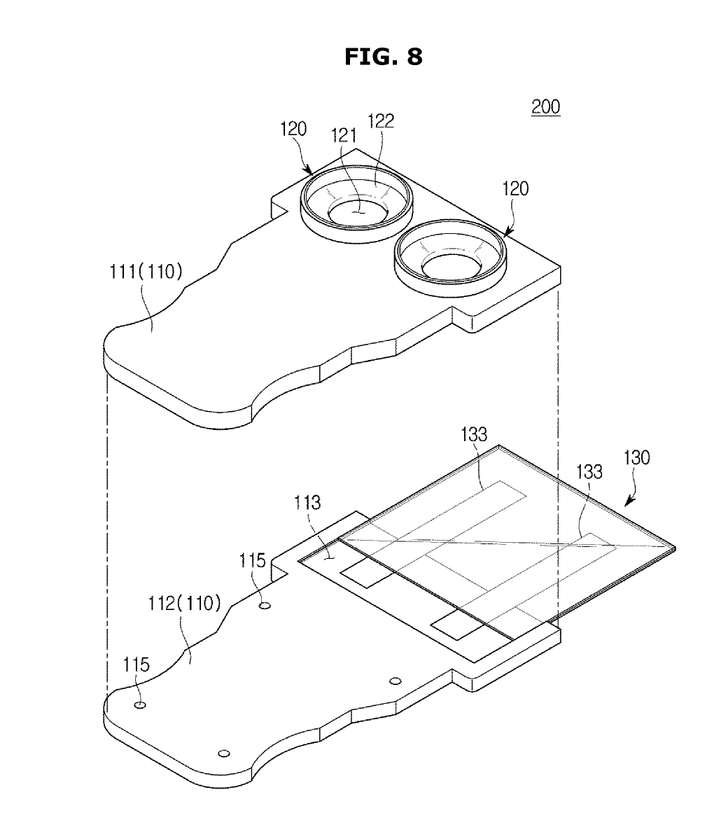

FIG. 7 is a perspective view of a sample analysis cartridge according to a second embodiment, and FIG. 8 is an exploded perspective view of the sample analysis cartridge according to the second embodiment. Hereinafter, descriptions overlapping with those described above with reference to FIGS. 1 to 5 will be omitted.

As shown in FIGS. 7 and 8, a sample analysis cartridge 200 may include at least two sample suppliers 120. For example, the sample analysis cartridge 200 may include a plurality of sample suppliers 120 that are adjacent to each other.

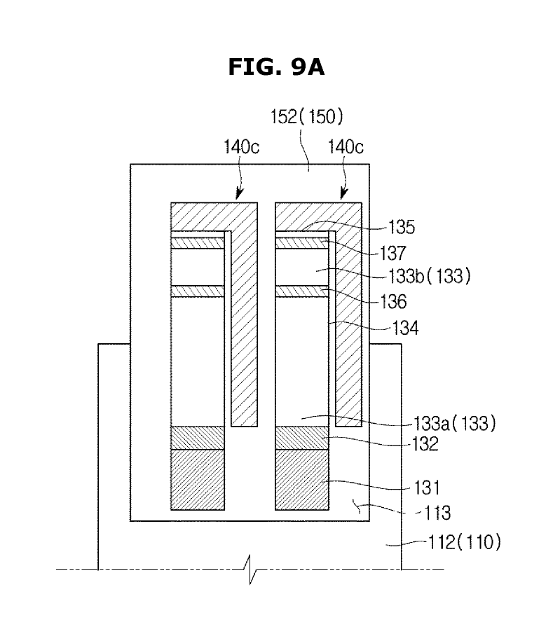

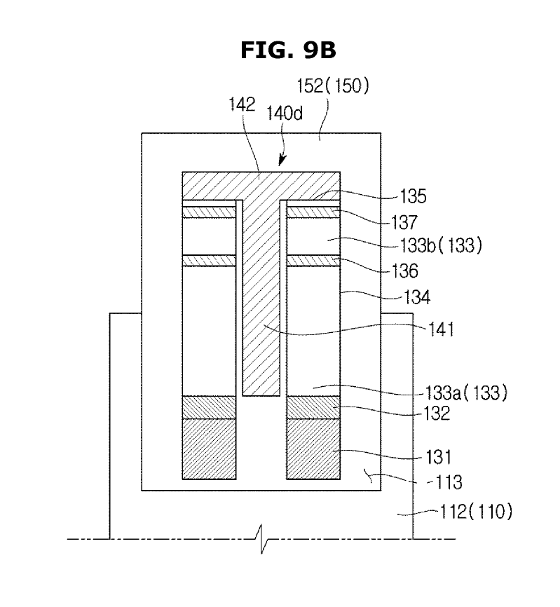

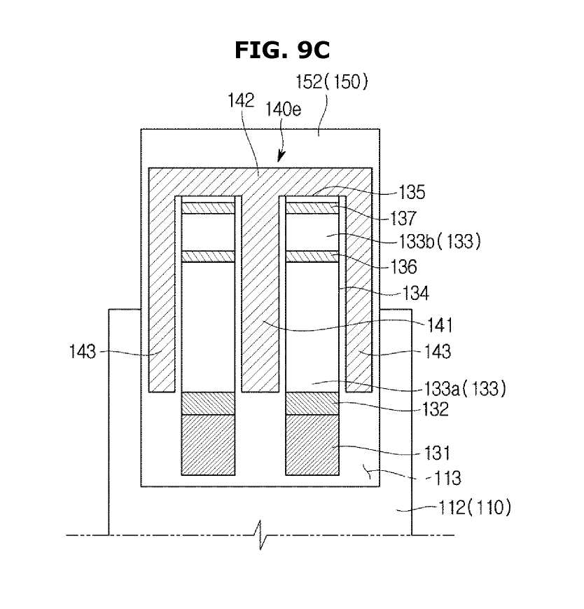

The sample analysis cartridge 200 may further include a strip 130a into which a sample entered through the at least two sample supplier 120 is supplied. The strip 130a may include at least two membranes 133 disposed to correspond to the at least two sample suppliers 120. The strip 130a may include a plurality of membranes 133 adjacent to each other. Also, as shown in FIGS. 9A, 9B, and 9C, the strip 130a may further include a binding pad 132 provided on the at least one membrane 133, and a sample pad 131 disposed on the binding pad 132. Also, the strip 130a may further include a cover 150 for supporting the at least one membrane 133. FIGS. 7 and 8 show two such membranes 133. The plurality of membranes 133 may be supported by a top cover 151 and a base cover 152.

As such, if the sample analysis cartridge 200 includes the plurality of sample suppliers 120 and the plurality of membranes 133, a plurality of tests may be simultaneously performed on a plurality of different samples in the sample analysis cartridge 200. The plurality of different samples may be the same kind of samples acquired from different sources. Also, the plurality of different samples may be different kinds of samples acquired from different sources. Also, the plurality of different samples may be the same kind of samples obtained from the same source, but being in different states.

FIGS. 9A, 9B, and 9C show various arrangements of an absorptive pad, in the sample analysis cartridge according to the second embodiment. Hereinafter, descriptions overlapping with those described above with reference to FIGS. 1 to 5 will be omitted.

As shown in FIG. 9A, the strip 130a may further include a plurality of absorptive pads 140c. Each of the plurality of absorptive pads 140c may surround a plurality of corners of each of the plurality of membranes 133. More specifically, each absorptive pad 140c may be disposed outside a shorter side 135 and a longer side 134 of each membrane 133. More specifically, each absorptive pad 140c may be disposed outside a shorter side 135 and a longer side 134 of the second end 133b of each membrane 133. The plurality of absorptive pads 140c may be spaced apart from each other without interfering with each other.

As shown in FIG. 9B, the strip 130a may further include an absorptive pad 140d disposed between the plurality of membranes 133. The absorptive pad 140d may include a first area 141 disposed between the plurality of membranes 133, and a second area 142 facing the shorter sides 135 of the second ends 133b of the plurality of membranes 133. The second area 142 may extend from the first area 141. For example, the absorptive pad 140d may be formed in a “T” shape. At least one of the first area 141 and the second area 142 of the absorptive pad 140d may contact the plurality of membranes 133.

As shown in FIG. 9C, the strip 130a may further include an absorptive pad 140e disposed between the plurality of membranes 133. The absorptive pad 140e may include a first area 141 disposed between the plurality of membranes 133, a second area 142 extending from the first area 141 to face the shorter sides 135 of the second ends 133b of the plurality of membranes 133, and a third area 143 extending from the second area 142 to face the outer longer sides 134 of the plurality of membranes 133. At least one of the first area 141, the second area 142, and the third area 143 of the absorptive pad 140e may contact the plurality of membranes 133.

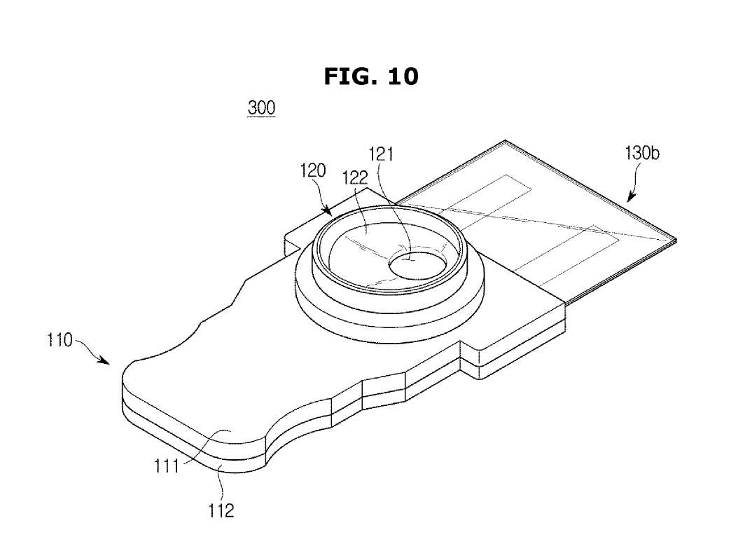

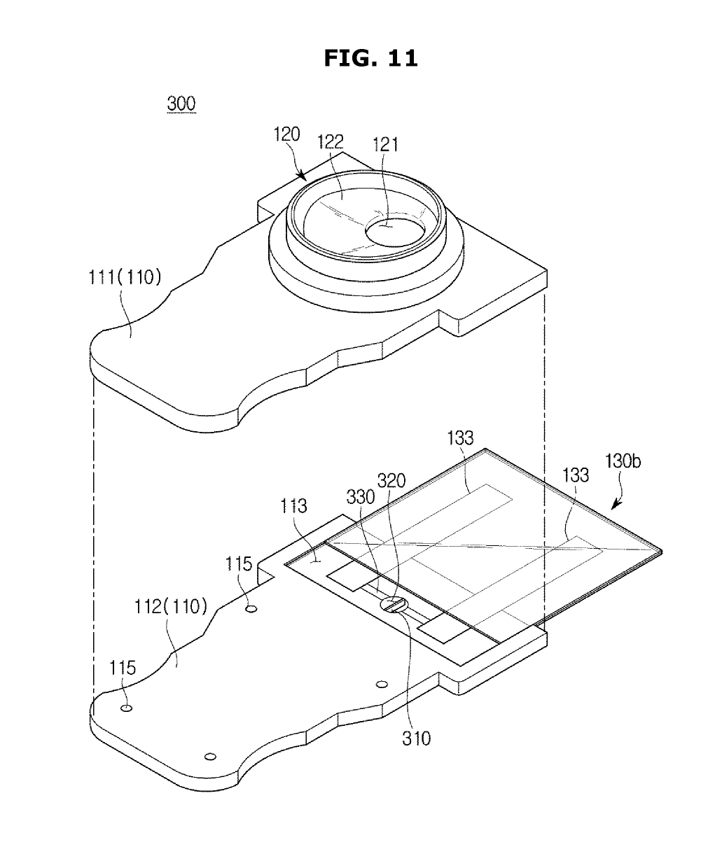

FIG. 10 is a perspective view of a sample analysis cartridge according to a third embodiment, and FIG. 11 is an exploded perspective view of the sample analysis cartridge according to the third embodiment. Hereinafter, descriptions overlapping with those described above with reference to FIGS. 1 to 5 will be omitted. Also, in the following description, a reference numeral “130b” indicates a strip.

As shown in FIGS. 10 and 11, a sample analysis cartridge 300 may include a plurality of membranes 133 disposed to respectively correspond to a plurality of sample accommodating spaces which will be described later.

The housing 110 may include a sample accommodating portion 320 formed in an inner surface of the housing 110 to accommodate a sample entered through the sample injecting hole 121. More specifically, the sample accommodating portion 320 may be recessed in the inner surface of the second frame 112 of the housing 110. The sample entered through the sample injecting hole 121 may be first accommodated in the sample accommodating portion 320, and then supplied to the plurality of membranes 133 along a sample moving channel 330 which will be described later.

The sample accommodating portion 320 may be partitioned to the plurality of sample accommodating spaces by a partition wall 310. That is, the partition wall 310 may protrude from the inner surface of the second frame 112 of the housing 110 to partition the sample accommodating portion 320 to the plurality of sample accommodating spaces.

The housing 110 may further include the sample moving channel 330 connecting the plurality of sample accommodating spaces to the plurality of membranes 133, respectively, to transfer samples accommodated in the sample accommodating portion 320 to the respective membranes 133. More specifically, the sample moving channel 330 may be recessed in the inner surface of the second frame 112 of the housing 110, adjacent to the sample accommodating portion 320.

The sample moving channel 330 and the sample accommodating portion 320 partitioned to the plurality of sample accommodating spaces by the partition wall 310 may be formed in the resting portion 113.

As such, if the sample analysis cartridge 300 includes the plurality of membranes 133 and the sample accommodating portion 320 partitioned to the plurality of sample accommodating spaces, a plurality of different tests can be performed on a sample.

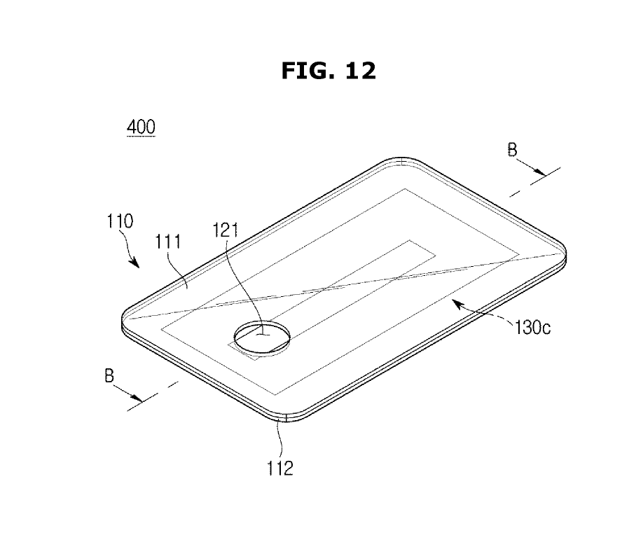

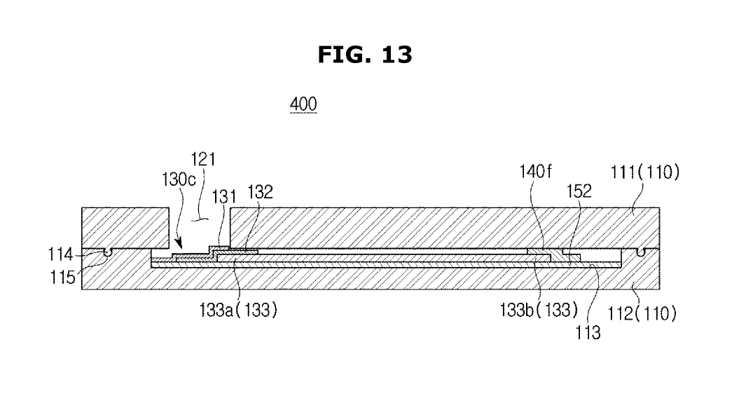

FIG. 12 is a perspective view of a sample analysis cartridge according to a fourth embodiment, and FIG. 13 is a cross-sectional view of the sample analysis cartridge according to the fourth embodiment. Hereinafter, descriptions overlapping with those described above with reference to FIGS. 1 to 5 will be omitted. Also, in the following description, components of the same names as those described above with reference to FIGS. 1 to 5 will be assigned the same reference numerals. FIG. 13 is a cross-sectional view of the sample analysis cartridge of FIG. 12, taken along line B-B of FIG. 12.

As shown in FIGS. 12 and 13, a sample analysis cartridge 400 may include a housing 110.

The housing 110 may be made of a transparent material. More specifically, the housing 110 may be made of a transparent material having light transmittance.

The housing 110 may include a first frame 111, and a second frame 112 coupled with the first frame 111 to form an outer appearance of the housing 110.

On any one of the first frame 111 and the second frame 112, a coupling protrusion 114 may be formed. In the other one of the first frame 111 and the second frame 112, a coupling groove 115 corresponding to the coupling protrusion 114 may be formed. The coupling protrusion 114 may be coupled with the coupling groove 115 so that the first frame 111 can be coupled with the second frame 112.

However, the first frame 111 may be coupled with the second frame 112 by another method. For example, the first frame 111 may be coupled with the second frame 112 by an adhesive material or member. Also, the first frame 111 may be coupled with the second member by welding.

The housing 110 may further include a resting portion 113 on which the strip 130 can be rested. The resting portion 113 may be recessed in the inner surface of the housing 110. More specifically, the resting portion 113 may be a recessed portion that is recessed in the inner surface of the second frame 112.

The sample analysis cartridge 400 may further include at least one sample injecting hole 121 into which a sample is supplied. The at least one sample injecting hole 121 may be formed in the housing 110. More specifically, the at least one sample injecting hole 121 may be formed in the first frame 111 of the housing 110. The at least one sample injecting hole 121 may be in the shape of a circle. However, the at least one sample injecting hole 121 may have another shape. When the sample analysis cartridge 400 is inserted in the installation member 30, the at least one sample injecting hole 121 may be pressed by the pressing member 40.

The sample analysis cartridge 400 may further include a strip 130c into which a sample entered through the at least one sample injecting hole 121 is supplied. The strip 130c may be disposed in the inside of the housing 110. More specifically, the strip 130c may be disposed or rested on the resting portion 113 of the housing 110 to be located in the inside of the housing 110.

The strip 130c may detect a target material from a sample through an antigen-antibody reaction.

The strip 130c may include a sample pad 131 for absorbing the sample entered through the at least one sample injecting hole 121. If the sample analysis cartridge 400 has a plurality of sample injection holes 121, the strip 130c may include a plurality of sample pads 131 for absorbing samples entered through the respective sample injection holes 121. The sample pad 131 has been described above with reference to FIGS. 2 to 5, and accordingly, a further description thereof will be omitted.

The strip 130c may further include a binding pad 132 disposed on the sample pad 131 to transfer a sample absorbed in the sample pad 131. If the sample analysis cartridge 400 has a plurality of sample injecting holes 121, the strip 130c may include a plurality of binding pads 132 disposed to correspond to the plurality of sample injecting holes 121. The binding pad 132 has been described above with reference to FIGS. 2 to 5, and accordingly, a further description thereof will be omitted.

The strip 130c may further include at least one membrane 133 disposed to correspond to the at least one sample injecting hole 121. A sample supplied to the at least one membrane 133 may move along the at least one membrane 133 by a capillary phenomenon. The at least one membrane 133 may be disposed at the binding pad 132 to transfer the sample supplied to the binding pad 132. In other words, the at least one membrane 133 may contact the binding pad 132. The at least one membrane 133 may include a first end 133a located adjacent to the at least one sample injecting hole 121, and a second end 133b that is opposite to the first end 133a. The binding pad 132 may be applied on the first end 133a of the at least one membrane 133.

The at least one membrane 133 may include a test line 136 and a control line 137. The test line 136 and the control line 137 have been described above with reference to FIGS. 2 to 5, and accordingly, a further description thereof will be omitted.

The strip 130c may further include an absorptive pad 140f disposed adjacent the at least one membrane 133 to absorb a sample passed through the at least one membrane 133. More specifically, the absorptive pad 140f may overlap the at least one membrane 133 to absorb a sample passed through the at least one membrane 133. In other words, at least one area of the absorptive pad 140f may be disposed on the at least one membrane 133. The absorptive pad 140f may be made of a porous material to absorb the sample from the at least one membrane 133.

The strip 130c may further include a base cover 152 for supporting the at least one membrane 133. The base cover 152 may be disposed or rested on the resting portion 113 of the housing 110 to support the at least one membrane 133.

The base cover 152 may be made of a transparent material. The base cover 152 may be formed of a transparent material to transmit light. For example, the base cover 152 may be made of a PET film. However, the base cover 152 may be made of another material, instead of a PET film.

A structure of the sample analysis cartridge 400 will be described in detail, below. The strip 130c may be disposed in the inside of the housing 110. The base cover 152 of the strip 130c may be disposed or rested on the resting portion 113 of the second frame 112 of the housing 110. The at least one membrane 133 may be disposed on the base cover 152. The binding pad 132 and the sample pad 131 may be applied sequentially on the first end 133a of the at least one membrane 133. The absorptive pad 140f may be applied on the second end 133b of the at least one membrane 133. An area of the binding pad 132 may be disposed on the first end 133a of the at least one membrane 133, and the other area of the binding pad 132 may be disposed on the base cover 152. Also, an area of the sample pad 131 may be disposed on the binding pad 132, and the other area of the sample pad 131 may be disposed on the base cover 152. Also, an area of the absorptive pad 140f may be disposed on the second end 133b of the at least one membrane 133, and the other area of the absorptive pad 140f may be disposed on the base cover 152.

The sample accommodating portion 320 and the sample moving channel 330 shown in FIGS. 10 and 11 may also be applied to the sample analysis cartridge 400 according to the fourth embodiment, or the other embodiments disclosed herein.

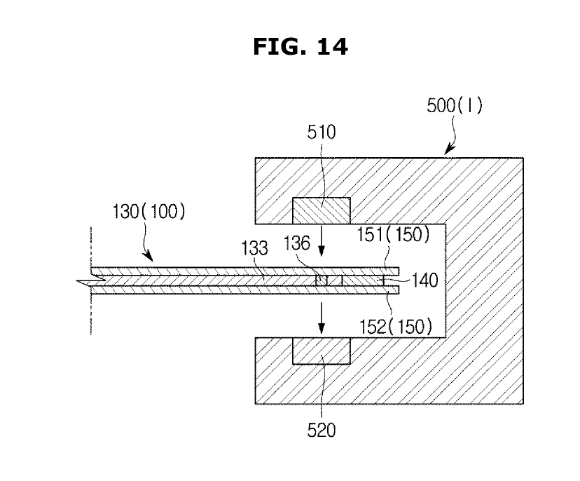

FIG. 14 is a view for briefly describing a process for measuring optical characteristics of the sample analysis cartridge according to the first embodiment. Hereinafter, a process for measuring optical characteristics of the sample analysis cartridge 100 according to the first embodiment will be described, however, the process for measuring optical characteristics of the sample analysis cartridge 100 can be applied in the same way to the sample analysis cartridge 200 according to the second embodiment, the sample analysis cartridge 300 according to the third embodiment, and the sample analysis cartridge 400 according to the fourth embodiment.

As shown in FIG. 14, the sample analysis apparatus 1 may further include an optical measurement module 500 configured to measure optical characteristics of the sample analysis cartridge 100. More specifically, the sample analysis apparatus 1 may further include the optical measurement module 500 configured to measure optical characteristics of the test line 136 of the sample analysis cartridge 100. The optical characteristics may be absorbance.

The optical measurement module 500 may include a light emitter 510 for radiating light to the test line 136, and a light receiver 520 for receiving light passed through the test line 136. The light emitter 510 may be opposite to the light receiver 520, and the sample analysis cartridge 100 may be positioned between the light emitter 510 and the light receiver 520. The light emitter 510 may include a light source such as a light emitting diode (LED). The light receiver may include an electronic device for measuring light such as a photodiode, image sensor, or camera. The optical measurement module 500 may also include processing hardware such as a controller, a memory, and a power source to process measured data.

The strip 130 may be made of a light transmitting material. More specifically, at least one area of the strip 130 corresponding to the test line 136 may be made of a light transmitting material.

The optical measurement module 500 may be used to optically detect a color represented on the test line 136 of the sample analysis cartridge 100, and to digitize the color. Measured data obtained by the optical measurement module 500 may be used to determine whether a target material exists in the corresponding sample, or a proportion of the target material.

As such, by forming at least one area of the strip 130 corresponding to the test line 136 with a light transmitting material, that is, a transparent material, the optical measurement module 500 can easily measure optical characteristics of the test line 136. Accordingly, quantitative analysis, as well as qualitative analysis, may be performed on a sample supplied to the sample analysis cartridge 100.

As described above, by implementing the sample analysis cartridge including the strip for detecting a target material from a sample through an antigen-antibody reaction, infection by harmful factors, such as parasite or virus, can be quickly detected.

In addition, since the optical characteristics of a test line can be measured by the optical measurement module, quantitative analysis, as well as qualitative analysis, can be performed on a sample.

Although embodiments of the disclosure have been shown and described, it would be appreciated by those skilled in the art that changes may be made in these embodiments without departing from the principles and spirit of the disclosure, the scope of which is defined in the claims and their equivalents.

What is claimed is:

1. A sample analysis cartridge comprising:
a housing comprising a sample injecting hole; and
a strip coupled with the housing and arranged such that a sample that is passed through the sample injecting hole is directed into the strip, wherein the strip is configured to detect a target material from the sample through an antigen-antibody reaction,
wherein the strip comprises:
a membrane including a test line; and
a transparent cover disposed outside the membrane.

2. The sample analysis cartridge according to claim 1, wherein the housing further comprises:
a first frame in which the sample injecting hole is formed; and
a second frame coupled with the first frame,
wherein a portion of the strip is located between the first frame and the second frame.

3. The sample analysis cartridge according to claim 2, wherein the second frame includes a recessed portion on which the strip is disposed.

4. The sample analysis cartridge according to claim 1, wherein the strip further comprises an absorptive pad disposed around the membrane and is configured to absorb the sample that is passed through the membrane.

5. The sample analysis cartridge according to claim 4, wherein the membrane comprises:
a first end located adjacent to the sample injecting hole; and
a second end that is opposite to the first end,
wherein the absorptive pad is disposed around the second end of the membrane.

6. The sample analysis cartridge according to claim 1, wherein the strip comprises:
a sample pad facing the sample injecting hole, and configured to absorb the sample that is passed through the sample injecting hole; and
a binding pad disposed between the sample pad and the membrane, and having an indicator configured to be bound with the target material of the sample.

7. The sample analysis cartridge according to claim 6, wherein the indicator includes at least one from among a gold nanoparticle and a latex particle.

8. The sample analysis cartridge according to claim 1, wherein the housing further comprises a sample supplier protruding from a surface of the housing, and the sample injecting hole is formed in the sample supplier.

9. The sample analysis cartridge according to claim 1, wherein the strip comprises a plurality of membranes, and
the housing comprises:
a sample accommodating portion formed in an inner surface of the housing to accommodate the sample that is passed through the sample injecting hole; and
a sample moving channel connecting the sample accommodating portion to the plurality of membranes to transfer the sample accommodated in the sample accommodating portion to the respective membranes.

10. The sample analysis cartridge according to claim 9, wherein the sample accommodating portion includes a partition wall that partitions the sample accommodating portion into a plurality of sample accommodating spaces.

11. The sample analysis cartridge according to claim 10, wherein the partition wall protrudes from the inner surface of the housing.

12. A sample analysis cartridge comprising:
a transparent housing comprising a sample injecting hole; and
a strip disposed inside of the transparent housing and arranged such that a sample that is passed through the sample injecting hole is directed to the strip, wherein the strip is configured to detect a target material from the sample through an antigen-antibody reaction.

13. The sample analysis cartridge according to claim 12, wherein the transparent housing further comprises:
a first frame in which the sample injecting hole is formed;
a second frame coupled with the first frame and comprising a recessed portion on which the strip is disposed.

14. The sample analysis cartridge according to claim 12, wherein the strip comprises:
a membrane including a test line; and
a transparent base cover configured to support the membrane.

15. The sample analysis cartridge according to claim 14, wherein the strip further comprises an absorptive pad overlapping the membrane, and configured to absorb the sample that is passed through the membrane.

16. The sample analysis cartridge according to claim 14, wherein the strip further comprises:
a sample pad facing the sample injecting hole, and configured to absorb the sample that is passed through the sample injecting hole; and
a binding pad disposed between the sample pad and the membrane, and having an indicator that can bind with the target material of the sample.

17. The sample analysis cartridge according to claim 16, wherein the indicator is at least one from among a gold nanoparticle and a latex particle.

18. A sample analysis apparatus comprising:
a sample analysis cartridge comprising:
a housing comprising at least one sample injecting hole, and
a strip coupled with the housing such that a sample that is passed through the sample injecting hole is directed into the strip, wherein the strip includes a test line configured to detect a target material from the sample through an antigen-antibody reaction;
a pressing member configured to press and seal off the at least one sample injecting hole; and
an optical measurement module configured to measure optical characteristics of the test line.

19. The sample analysis apparatus according to claim 18, wherein the optical measurement module comprises:
a light emitter configured to radiate light to the test line; and
a light receiver configured to receive the light that passes through the test line,
wherein at least one area of the strip corresponding to the test line is made of a light transmitting material.

20. The sample analysis apparatus according to claim 18, wherein the strip comprises:
at least one membrane disposed to correspond to the at least one sample injecting hole, and including the test line;
an absorptive pad disposed around the at least one membrane, and configured to absorb the sample that is passed through the at least one membrane; and
a transparent cover disposed outside the at least one membrane and the absorptive pad, and configured to support the at least one membrane and the absorptive pad.
